(19) United States
(12) Patent Application Publication
(10) Pub. No.: US 2020/0283001 A1
(43) Pub. Date: Sep. 10, 2020

(54) VEHICULAR DRIVER MONITORING SYSTEM USING BREATH SENSOR

(71) Applicant: Magna Electronics Inc., Auburn Hills, MI (US)

(72) Inventor: Gaurav Kulkarni, Pune (IN)

(21) Appl. No.: 16/808,946

(22) Filed: Mar. 4, 2020

Related U.S. Application Data
(60) Provisional application No. 62/813,312, filed on Mar. 4, 2019.

(51) Int. Cl. B60W 40/08; A61B 5/08; B60W 50/14

(52) U.S. Cl. CPC B60W 40/08; A61B 5/0809; B60W 2050/143; B60W 2040/0818; B60W 2040/0872; B60W 50/14

(57) ABSTRACT

A vehicular driver monitoring system includes a pneumographic sensor disposed in a cabin of a vehicle and operable to capture sensor data indicative of breathing of a driver of the vehicle. The pneumographic sensor measures an aspect associated with breathing of the driver. A control includes electronic circuitry and associated software, with the electronic circuitry including a data processor operable to process sensor data captured by the pneumographic sensor. The control, responsive to processing of sensor data captured by the pneumographic sensor, monitors the driver based on the measured aspect associated with the breathing of the driver.

### CROSS REFERENCE TO RELATED APPLICATION

The present application claims the filing benefits of U.S. provisional application Ser. No. 62/813,312, filed Mar. 4, 2019, which is hereby incorporated herein by reference in its entirety.

### FIELD OF THE INVENTION

The present invention relates generally to a vehicle driver monitoring system for a vehicle and, more particularly, to a vehicle driver monitoring system that utilizes one or more sensors at a vehicle.

### BACKGROUND OF THE INVENTION

Monitoring a driver of a vehicle for inattention and fatigue using imaging sensors is known.

### SUMMARY OF THE INVENTION

The present invention provides a driver assistance system or driver monitoring system for a vehicle that utilizes one or more wearable sensors to capture sensor data. The captured sensor data is processed by a processor, and the processor, in response to processing the sensor data, determines inattention or fatigue of the driver. The wearable sensor may be a textile impedance based pneumography sensor that measures or senses a breathing or respiratory rate of the driver. The processor may wirelessly communicate captured sensor data to a tracking device (e.g., via BLUETOOTH or other suitable short range communication protocol).

These and other objects, advantages, purposes and features of the present invention will become apparent upon review of the following specification in conjunction with the drawings.

### BRIEF DESCRIPTION OF THE DRAWINGS

FIG. 1 is a plan view of a vehicle with a driver monitoring system that incorporates sensors in accordance with the present invention;

FIG. 2 is schematic of a circuit with a condenser microphone to measure breath;

FIG. 3 is a graph of the measurements of the microphone of FIG. 2;

FIG. 4 is a schematic of a circuit with impedance based pneumographic devices in accordance with the present invention;

FIG. 5 is a cross section of a textile impedance based pneumography sensor in accordance with the present invention;

FIG. 6 is a plot of different breathing types as measured by the circuit of FIG. 4;

FIGS. 7A-7C are perspective views of sensor contact with a human body through different breathing states; and

FIG. 8 is a block diagram of a driver monitoring system in accordance with the present invention.

### DESCRIPTION OF THE PREFERRED EMBODIMENTS

A driver monitoring system and/or driver assist system and/or advanced driver-assistance system and/or alert system operates to monitor a driver for distraction or fatigue using wearable sensors that measure biological signals. The driver monitoring system includes a processor or processing system that is operable to receive sensor data from one or more sensors and provide an output to a system of the vehicle, a device of the driver, a remote server, a display device, etc.

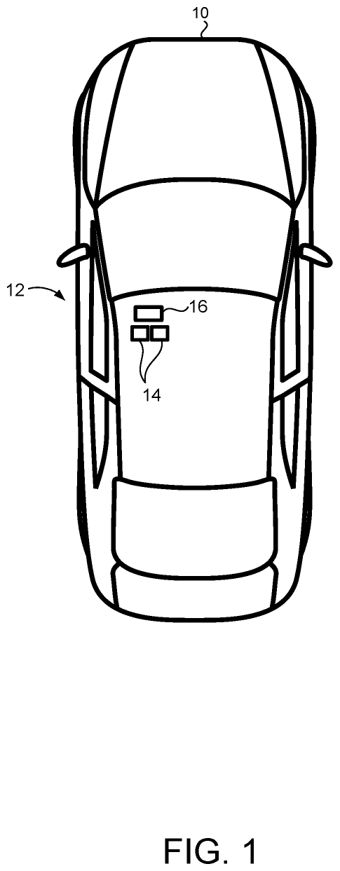

Referring now to the drawings and the illustrative embodiments depicted therein, a vehicle 10 includes a driver monitoring system 12 that includes at least one sensor 14, such as a pneumographic sensor, such as, for example, an impedance based pneumography sensor, such as, for example, a textile impedance pneumography sensor (FIG. 1). The driver monitoring system 12 includes a control or electronic control unit (ECU) 16 having electronic circuitry and associated software, with the electronic circuitry including a data processor that is operable to process sensor data captured by the sensor or sensors. The ECU may process sensor data to detect driver fatigue or inattention. The data transfer or signal communication from the sensor 14 to the ECU may comprise any suitable data or communication link, such as wireless communication or a vehicle network bus or the like of the equipped vehicle.

Modern vehicles provide many ways to control devices, such as touch, voice recognition, remote control, and gestures. Many modern vehicles also provide driver inattention monitoring. For example, the vehicle may monitor for driver fatigue and alert the driver with a suggestion to rest when fatigue is detected. Driver inattention may be generally classified into two types: distraction and fatigue. Driver inattention is a major factor in most traffic accidents. Research and development has actively been carried out for decades, with the goal of precisely determining the drivers' state of mind. This research has led to sensing biological signals such as with electroencephalograms (EEG), electrocardiograms (ECG), electro-oculography (EOG), and surface electromyogram (sEMG). These signals are collected through wearable sensors and the collected data may be processed for analysis. The goal is to develop an efficient algorithm which can analyze biological signals and determine appropriate action in response. Current driver inattention monitoring systems typically use face detection (e.g., via an interior camera viewing the driver's head region), eye detection, and other techniques which are costly due to expensive hardware cost (more powerful microprocessors, more memory, complex hardware such as cameras, etc.) and software efforts and also due to increases in time to market and development costs. Sensors for sensing biological signals (e.g., pneumography sensors) do not use costly video cameras and face recognition systems and are typically less expensive, and thus these sensors may be more popular in cost sensitive markets. Also, many driver inattention systems monitor characteristics of the driving environment (e.g., the type of road, weather conditions, and traffic density) instead of the driver.

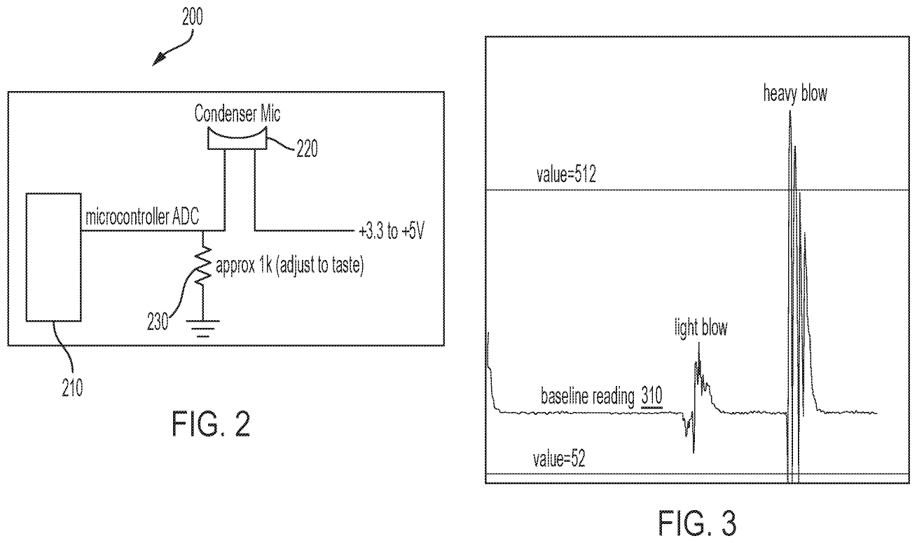

Referring now to FIG. 2, the driver monitoring system 12 may include a circuit 200 with a microcontroller 210. The microcontroller 210 may include an ADC channel input connected to a microphone 220 (e.g., a unidirectional condenser microphone). The microcontroller 210 may receive a signal from the microphone 220 and determine a reading based on a voltage of the signal and a value of a resistor 230. For example, the resistor value may be 1000 ohms. The microphone may be provided with a voltage (e.g., between 3.3V and 5V). The microphone measures sound levels (e.g., sounds of the driver breathing). As illustrated in FIG. 3, the value measured by the microcontroller 210 fluctuates as the microphone 220 detects changes in breathing. For example, the microphone may have a baseline reading 310 when no breathing is detected, a small peak when a light blow or breath is detected, and a much larger peak when a heavy blow or breath is detected. That is, the heavier the breathing of the driver, the larger the amplitude of the signal captured by the microphone 220. The breath may need to exceed a threshold value (e.g., 512 as illustrated) to trigger the sensor and/or ADC and/or control. The circuit 200 may not be feasible for actual application in some vehicles (e.g., due to positioning the microphone, etc.). Instead, a textile based capacitive breath sensing system is provided.

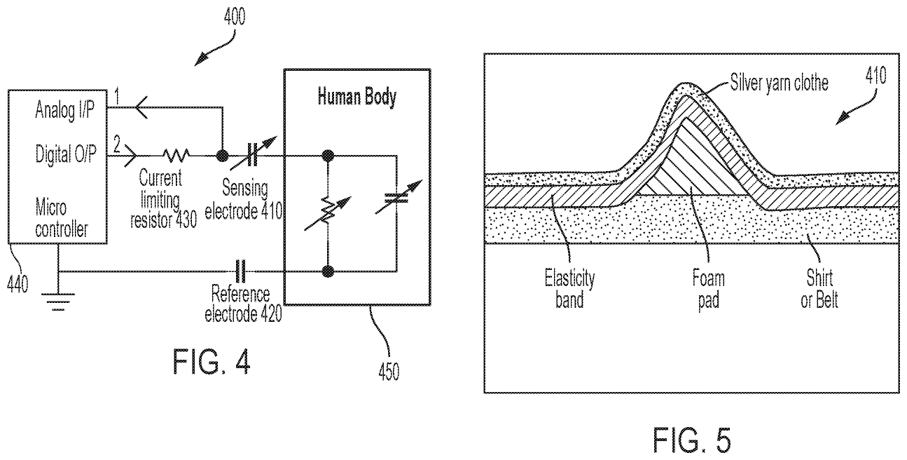

Impedance pneumography is a known technique to measure respiration rate or breathing rate in clinical operation. This technique gives reliable results by conventional patches (i.e., by attaching two or four electrodes to the user's chest using, for example, a flexible rubber vessel) to measure changes in the electrical impedance of the user's thorax caused by respiration or breathing. Typical electrodes (i.e., electrodes applied directly to skin, generally using a gel) are neither comfortable nor feasible for a wearable system in a vehicle. To increase comfort, wearability, and effectiveness of the wearable system, the most desirable sensor forms are fabrics/textiles themselves, i.e., textile-based sensors. Such textiles-based sensors may accurately detect breath rate while maintaining comfort and wearability in a vehicle. The human body may be modeled as parallel connected variable capacitor and resistors (FIG. 4). In accordance with the present invention, the driver monitoring system 12 may charge this capacitor (i.e., an occupant of the vehicle) to provide an analog breathing curve, as impedance pneumography does.

Some pneumographic devices are impedance based. These devices use a high frequency (tens to hundreds of kHz) and low amplitude current that is injected across the chest cavity. The voltage resulting from this current injection is measured and the resistance is derived from the application of Ohm's law (R=V/I). Because current flows less easily through the chest as the lungs fill with air, the resistance rises with increasing lung volume. This increase in resistance is measured and can be correlated to the user's breathing rate or pattern.

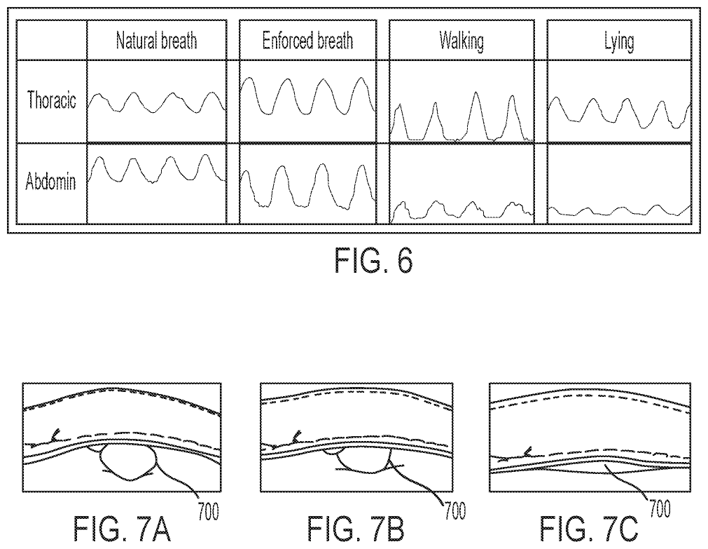

Referring now to FIG. 4, an impedance based circuit 400 includes a sensing electrode 410, a reference electrode 320, a, for example, 10K Ohms current limiting resistor 430, and a microcontroller 440. As previously discussed, a model 450 of the human body is illustrated as a parallel connected variable capacitor and resistor. As shown in FIG. 5, the sensing electrode 410 may be a three-dimensional (3D) triangle shape with conducting clothes or other textiles fixed at both sides. This electrode 410 may lay on top of the driver's shirt or belt. In some implementations, the electrodes 410, 420 may be disposed at, integrated with, or attached to the vehicle's seatbelt. Because the human body acts like the equivalent circuit of a combination of a capacitor and a resistor, where the capacitance and resistance are varied by the body movements (e.g., breathing), both electrodes may be modeled as capacitors when loosely contacting a human body. The textile sensor 400 (i.e., the reference electrode, the sensing electrode, and the microcontroller 440) may discern differences in the driver's breathing. For example, FIG. 6 illustrates different breathing patterns of thoracic (i.e., measured at the thorax) and abdomen (i.e., measured at the abdomen) breathing for natural breathing, enforced breathing, breathing while walking, and breathing while lying down.

When exhaling, the body shrinks inward and the contact area of an electrode 700 decreases (FIG. 7A). That is, the amount of surface area of the electrode 700 in contact with the occupant decreases. When inhaling, the thorax expands and the contact area increases. FIG. 7B illustrates thorax expansion and increased electrode contact area during natural (not forced) inhaling and with FIG. 7C showing thorax expansion and increased electrode contact area during forced inhaling (e.g., drawing in a deep breath). Because surface area affects impedance values, the charging impedance is thus varied according to breathing movement. To measure the charging impedance, the microcontroller may assign a logic ‘1’ to the charging end to charge the human capacitor. When the voltage on the sensing electrode rises to a pre-determined threshold value, the microcontroller switches the charging end to logic ‘0’ (e.g., ground) to discharge the human capacitor. Thus the microcontroller may obtain continuous digital data of charging time, which reflects the breathing movement (i.e., impedance pneumography).

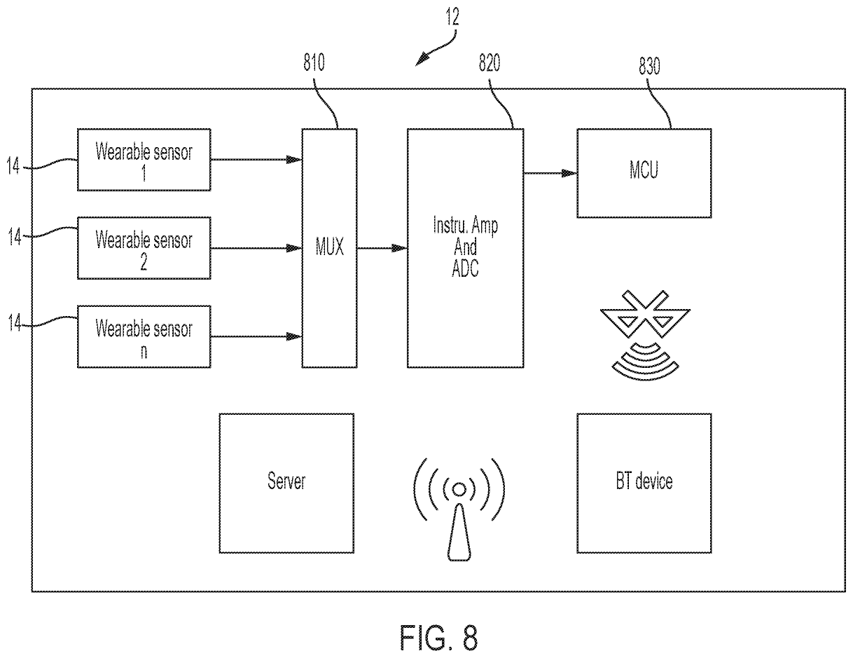

The driver monitoring system 12 may be useful to a telematics or driver assistance system. For example, the system 12 may raise an alarm if it detects the driver is sleepy, has fallen asleep, or has fallen unconscious. Additionally, the cost is low (especially compared against, for example, vision-based systems) while remaining reliable. Referring now to FIG. 8, the driver monitoring system 12 may include a plurality of wearable sensors 14 that are used to detect biological signals from driver (e.g., for detecting distraction or fatigue). These signals are fed through an optional multiplexor 810 and amplifier 820 to the microcontroller 830 which may convert the signals to a human readable format. These values are sent to a wireless (e.g., BLUETOOTH) tracking device via wireless communication (e.g., BLUETOOTH Low Energy (BLE), BLUETOOTH (BT), or Wi-Fi) and further shared with a server for analysis and action. Alternatively, the values may be transmitted along a wire (e.g., built in to a seatbelt) to, for example, the ECU 16 of the vehicle. The ECU 16 may store the data in non-volatile storage and/or wirelessly transmit the data to a remote location (e.g., a data server). The wearable sensors may detect EEG, ECG, and more parameters which assist in understanding the current health and status of driver. When appropriate, the system may warn the driver or request the driver to stop and rest or take other action to reduce fatigue or distraction. The system may include an extension of a BLUETOOTH device with no extra investment required. Sensors may be integrated with textiles without requiring gel or other connection enhancements for connection with a human body. Thus, the total integration of the sensing elements and connections into the garment presents great advantages in physical as well as psychological comfort for the user. Further, information gathered by the system may be further used by the driver or fleet owner as desired.

The system provides a low cost alternative to other driver assistance systems and the system may utilize radio frequency (RF) or BLE. The system may be powered via a battery with a low battery consumption which allows for a long life. The system may also be powered, for example, along the seatbelt. The system is convenient for current consumers and shortens time to market. The system may, in some examples, be an extension of BT devices, and thus may be an extension of a current advanced driver-assistance systems (ADAS) platform.

Changes and modifications in the specifically described embodiments can be carried out without departing from the principles of the invention, which is intended to be limited only by the scope of the appended claims, as interpreted according to the principles of patent law including the doctrine of equivalents.

1. A vehicular driver monitoring system, the vehicular driver monitoring system comprising:
a pneumographic sensor disposed in a cabin of a vehicle and operable to capture sensor data measuring an aspect associated with breathing of a driver of the vehicle;
a control comprising electronic circuitry and associated software, wherein the electronic circuitry comprises a processor operable to process sensor data captured by the pneumographic sensor and provided to the control; and
wherein the control, responsive to processing by the processor of sensor data captured by the pneumographic sensor, monitors the driver based on the measured aspect associated with the breathing of the driver.

2. The vehicular driver monitoring system of claim 1, wherein the pneumographic sensor is integrated into a seatbelt of the vehicle.

3. The vehicular driver monitoring system of claim 1, wherein the pneumographic sensor comprises an impedance based pneumography sensor.

4. The vehicular driver monitoring system of claim 3, wherein the impedance based pneumography sensor comprises a textile impedance based pneumography sensor.

5. The vehicular driver monitoring system of claim 1, wherein the pneumographic sensor is operable to measure a breathing rate of the driver.

6. The vehicular driver monitoring system of claim 5, wherein the pneumographic sensor is operable to measure an electrical impedance of the driver's thorax.

7. The vehicular driver monitoring system of claim 1, wherein the pneumographic sensor comprises a sensing electrode disposed at the driver's thorax or abdomen and a reference electrode disposed at the driver's thorax or abdomen, and wherein the reference electrode transmits a signal through the driver's thorax or abdomen, and wherein the sensing electrode receives the transmitted signal through the driver's thorax or abdomen.

8. The vehicular driver monitoring system of claim 1, wherein an amount of contact of the pneumographic sensor with the driver changes based on the driver's breathing.

9. The vehicular driver monitoring system of claim 8, wherein the amount of contact of the pneumographic sensor with the driver increases when the driver inhales, and wherein the amount of contact of the pneumographic sensor with the driver decreases when the driver exhales.

10. The vehicular driver monitoring system of claim 9, wherein the pneumographic sensor comprises an impedance based pneumography sensor, and wherein the impedance measured by the pneumographic sensor changes is based on the amount of contact of the pneumographic sensor with the driver.

11. The vehicular driver monitoring system of claim 1, wherein the processor wirelessly communicates the processed sensor data to a tracking device.

12. The vehicular driver monitoring system of claim 11, wherein the processor wirelessly communicates with the tracking device via a BLUETOOTH communication link.

13. The vehicular driver monitoring system of claim 11, wherein the tracking device wirelessly communicates the processed sensor data to a remote server.

14. The vehicular driver monitoring system of claim 1, wherein the control, responsive to processing by the processor of sensor data captured by the pneumographic sensor, determines inattention of the driver based on the measured aspect associated with the breathing of the driver.

15. The vehicular driver monitoring system of claim 1, wherein the control, responsive to processing by the processor of sensor data captured by the pneumographic sensor, determines fatigue of the driver based on the measured aspect associated with the breathing of the driver.

16. A vehicular driver monitoring system, the vehicular driver monitoring system comprising:
a textile impedance based pneumography sensor disposed in a cabin of a vehicle and operable to capture sensor data measuring a breathing rate of a driver of the vehicle;
a control comprising electronic circuitry and associated software, wherein the electronic circuitry comprises a processor operable to process sensor data captured by the textile impedance based pneumography sensor and provided to the control; and
wherein the control, responsive to processing by the processor of sensor data captured by the textile impedance based pneumography sensor, monitors the driver based on the measured breathing rate of the driver to determine at least one selected from the group consisting of (i) driver attentiveness and (ii) driver drowsiness.

17. The vehicular driver monitoring system of claim 16, wherein the textile impedance based pneumography sensor comprises a sensing electrode disposed at the driver's thorax or abdomen and a reference electrode disposed at the driver's thorax or abdomen, and wherein the reference electrode transmits a signal through the driver's thorax or abdomen, and wherein the sensing electrode receives the transmitted signal through the driver's thorax or abdomen.

18. The vehicular driver monitoring system of claim 16, wherein an amount of contact of the textile impedance based pneumography sensor with the driver changes based on the driver's breathing.

19. A vehicular driver monitoring system, the vehicular driver monitoring system comprising:
a textile impedance based pneumography sensor disposed in a cabin of a vehicle and operable to capture sensor data measuring an aspect associated with breathing of a driver of the vehicle;
wherein the textile impedance based pneumography sensor comprises a sensing electrode disposed at the driver's thorax or abdomen and a reference electrode disposed at the driver's thorax or abdomen, and wherein the reference electrode transmits a signal through the driver's thorax or abdomen, and wherein the sensing electrode receives the transmitted signal through the driver's thorax or abdomen
a control comprising electronic circuitry and associated software, wherein the electronic circuitry comprises a processor operable to process sensor data captured by the textile impedance based pneumography sensor and provided to the control; and
wherein the control, responsive to processing by the processor of sensor data captured by the textile impedance based pneumography sensor, monitors the driver based on the measured aspect associated with breathing of the driver of the vehicle.

20. The vehicular driver monitoring system of claim 19, wherein an amount of contact of the textile impedance based pneumography sensor with the driver increases when the driver inhales, and wherein the amount of contact of the textile impedance based pneumography sensor with the driver decreases when the driver exhales.
